(19) United States
(12) Patent Application Publication
(10) Pub. No.: US 2008/0058572 A1
(43) Pub. Date: Mar. 6, 2008

(54) PROCESS FOR PREPARING ISOOLEFINS

(75) Inventors: Silvia Santiago Fernandez, Oviedo (ES); Markus Winterberg, Datteln (DE); Franz Nierlich, Marl (DE); Stephan Houbrechts, Duffel (BE); Horst-Werner Zanthoff, Muelheim a.d. Ruhr (DE); Wilfried Bueschken, Haltern am See (DE); Walter Luh, Marl (DE); Georg Skillas, Hanau (DE)

Correspondence Address: OBLON, SPIVAK, MCCLELLAND MAIER & NEUSTADT, P.C., 1940 DUKE STREET, ALEXANDRIA, VA, 22314, US

(73) Assignee: OXENO OLEFINCHEMIE GMBH, Marl (DE)

(21) Appl. No.: 11/839,597

(22) Filed: Aug. 16, 2007

(30) Foreign Application Priority Data
Aug. 29, 2006 (DE) 102006040433.5

(51) Int. Cl. C07C 1/20

(52) U.S. Cl. 585/640; 585/639

(57) ABSTRACT

A continuous process for preparing an isoolefin having 4 to 6 carbon atoms is performed by cleaving a compound of the formula I

R₁—O—R₂ (I)

wherein R₁=a tertiary alkyl radical having 4 to 6 carbon atoms, and R₂=H or an alkyl radical, in a gas phase over a solid catalyst, in the temperature range of 200 to 400° C., at a pressure of 0.1 to 1.2 MPa, in a reactor which is equipped with a heating jacket and is heated with a liquid heat carrier, wherein the cleavage is carried out in such a way that a temperature drop in the catalyst zone at any point in relation to the entrance temperature is less than 50° C., wherein (i) a reaction mixture in the reactor and (ii) the heat carrier in the jacket flow through the reactor in cocurrent, and wherein a temperature difference of the heat carrier between a feed point to the reactor and an outlet from the reactor is adjusted to less than 40° C.

### BACKGROUND OF THE INVENTION

1. Field of the Invention

The present invention relates to a process for preparing an isoolefin.

2. Discussion of the Background

Isoolefins, for example isobutene, are important intermediates for the preparation of a multitude of organic compounds. Isobutene is, for example, a starting material for the preparation of a multitude of products, for example for the preparation of butyl rubber, polyisobutylene, isobutene oligomers, branched C₅ aldehydes, C₅ carboxylic acids, C₅ alcohols and C₅ olefins. It is also used as an alkylating agent, especially for the synthesis of tert-butylaromatics, and as an intermediate for obtaining peroxides. In addition, isobutene can be used as a precursor for the preparation of methacrylic acid and esters thereof.

In industrial streams, isoolefins are usually present together with other olefins and saturated hydrocarbons with the same or a different number of carbon atoms. Especially from mixtures which comprise isoolefins together with other olefins and saturated hydrocarbons with the same number of carbon atoms per molecule, it is not possible to remove the isoolefins (in an economically viable manner) with physical separating methods alone. For example, isobutene is present in customary industrial streams together with saturated and unsaturated C₄ hydrocarbons. Isobutene cannot be removed in an economically viable manner from these mixtures by distillation owing to the low boiling point difference and the low separating factor between isobutene and 1-butene.

Isobutene is therefore typically obtained from technical hydrocarbon mixtures by converting isobutene to a derivative which can easily be removed from the remaining hydrocarbon mixture, and by cleaving the isolated derivative back to isobutene and derivatizing agent.

Typically, isobutene is removed from C₄ cuts, for example the C₄ fraction of a steamcracker, as follows. After removal of the majority of the polyunsaturated hydrocarbons, mainly butadiene, by extraction or extractive distillation or selective hydrogenation to linear butenes, the remaining mixture (raffinate I or hydrogenated crack-C₄) is reacted with alcohol or water. When methanol is used, methyl tert-butyl ether (MTBE) is formed from isobutene, and, when water is used, tert-butanol (TBA). After they have been removed, both products can be cleaved to give isobutene in a reversal of their formation.

MTBE is less expensive than TBA because the reaction of isobutenic hydrocarbons with methanol is easier than with water, and MTBE is produced in large amounts as a component of gasoline fuels. The recovery of isobutene from MTBE is therefore potentially more economically viable than that from TBA if a similarly good process to that for the cleavage of TBA were available for the cleavage of MTBE.

The cleavage of ethers having a tertiary alkyl radical to give the corresponding isoolefins and alcohols, and the cleavage of tertiary alcohols to give the corresponding isoolefins and water, can be performed in the presence of acidic catalysts in the gas phase or gas/liquid mixed phase or in the pure gas phase.

The cleavage in the liquid phase gas/liquid phase has the disadvantage that the products formed, dissolved in the liquid phase, can enter into side reactions. For example, the isobutene formed in the cleavage of MTBE forms undesired C₈ and C₁₂ components by acid-catalysed dimerization or oligomerization. The undesired C₈ components are mainly 2,4,4-trimethyl-1-pentene and 2,4,4-trimethyl-2-pentene. In addition, a portion of the methanol formed in the cleavage is convereted to dimethyl ether with elimination of water.

With increasing cleavage temperature, side reactions, for example hydrogenations or dehydrogenations, increase. In addition, the specific energy consumption rises with increasing temperature. Moreover, high cleavage temperatures entail a greater capital investment for the reactors. It is therefore appropriate to perform the cleavage of isoolefin derivatives at temperatures below 400° C.

Various processes for preparing isobutene by cleaving MTBE in the gas phase are known.

DE 102 27 350 and DE 102 27 351 describe processes for preparing isobutene by cleaving MTBE in the gas phase. In both processes, temperatures of 150 to 300° C. are used. U.S. Pat. No. 6,072,095 and U.S. Pat. No. 6,143,936 likewise describe processes for preparing isobutene by cleaving MTBE. In these processes, temperatures of 50 to 300° C., preferably of 100 to 250° C., are used.

A problem in the performance of the cleavage in the gas phase at relatively low temperatures is the relatively rapid deactivation of the catalyst.

Since the activity of catalysts decreases during operation, it is advantageous, in order to retain the conversion, to counteract it by increasing the temperature. In order to maintain operation with a catalyst for as long as possible, as low as possible a temperature within the intended temperature range is therefore desirable when fresh catalyst is used.

The cleavage of isoolefin derivatives is endothermic. In the cleavage of the isoolefin derivatives, a high temperature drop can therefore occur in the first catalyst zone. Starting from low entrance temperatures, this can lead to a particularly great drop in the temperature in the catalyst and to the catalyst becoming deactivated more rapidly or more greatly.

For the performance of endothermic reactions, reactors in which the heat of reaction is supplied internally or externally can be used. Reactors with internal heating (heating rods or plates or tubes which are flowed through by a heating medium) entail, owing to their complicated construction, a high capital investment. Reactors which are heated externally are usually a tubular reactor or tube bundle reactor. These are usually heated with the aid of a heat carrier which flows through a closed jacket which surrounds the tube or the tubes. In order to restrict the temperature drop in the reactor in endothermic reactions, it is possible, instead of one reactor, to use a plurality of reactors connected in series which are operated at different temperatures. It is also possible to use one reactor whose jacket is divided into different regions which can be charged with heat carrier at different temperatures. However, these constructions are complicated and require a high capital investment. In addition, the operating costs are higher than in the case of reaction in a reactor with only one heating circuit.

In order to reduce the temperature drop in the catalyst zone, it is also possible to use catalysts of different activity in one tubular reactor. For example, in a first catalyst zone, a catalyst having a lower activity than in the downstream zone can be used. Instead of different catalysts, it is also possible to use mixtures of one catalyst with different proportions of an inert material in the different zones. These procedures have the disadvantage that different catalysts have to be kept ready or different catalyst mixtures have to be prepared. Moreover, the layered filling of a tubular reactor with a plurality of catalysts or catalyst mixtures is more complicated than the filling with one catalyst.

### SUMMARY OF THE INVENTION

It was therefore an object of the present invention to provide an alternative process for the catalytic gas phase cleavage of isoolefin derivatives to isoolefin and alcohol or water in an inexpensive and/or simple apparatus in which only slight, if any, catalyst deactivation occurs.

This and other objects have been achieved by the present invention the first embodiment of which includes a continuous process for preparing an isoolefin having 4 to 6 carbon atoms by cleaving a compound of the formula I

R₁—O—R₂ (I)

wherein R₁=a tertiary alkyl radical having 4 to 6 carbon atoms, and

R₂═H or an alkyl radical,

in a gas phase over a solid catalyst, in the temperature range of 200 to 400° C., at a pressure of 0.1 to 1.2 MPa, in a reactor which is equipped with a heating jacket and is heated with a liquid heat carrier,

wherein said cleavage is carried out in such a way that a temperature drop in the catalyst zone at any point in relation to the entrance temperature is less than 50° C.,

wherein (i) a reaction mixture in the reactor and (ii) the heat carrier in the jacket flow through the reactor in cocurrent, and

wherein a temperature difference of the heat carrier between a feed point to the reactor and an outlet from the reactor is adjusted to less than 40° C.

### DETAILED DESCRIPTION OF THE INVENTION

The present invention relates to a process for preparing an isoolefin, especially isobutene, by cleaving an alkyl tert-alkyl ether or a tertiary alcohol. In particular, the present invention relates to a process for cleaving an alkyl tert-alkyl ether, especially MTBE, to isoolefin and alcohol.

It has now been found that, surprisingly, alkyl tert-alkyl ethers and tertiary alcohols can be cleaved in a simple manner to isoolefins having 4 to 6 carbon atoms and alcohol or water over a solid catalyst in the gas phase in the temperature range of 200 to 400° C. at a pressure of 0.1 to 1.2 MPa in a simple tubular reactor or tube bundle reactor which is heated with a liquid heat carrier without high catalyst deactivation being observed when the maximum temperature drop at any point in the catalyst zone is adjusted to less than 50° C. when the reaction mixture and the heat carrier flow in cocurrent (in separate spaces) through the reactor and the temperature difference of the heat carrier between feed point to the reactor and outlet from the reactor is less than 40° C.

The present invention therefore provides a continuous process for preparing isoolefins having 4 to 6 carbon atoms by cleaving compounds of the formula I

R₁—O—R₂ (I)

where R₁=a tertiary alkyl radical having 4 to 6 carbon atoms and R₂═H or an alkyl radical in the gas phase over a solid catalyst in the temperature range of 200 to 400° C. at a pressure of 0.1 to 1.2 MPa in a reactor which is equipped with a heating jacket and is heated with a liquid heat carrier, characterized in that the temperature drop in the catalyst zone at any point in relation to the entrance temperature is less than 50° C., in that the reaction mixture in the reactor and the heat carrier in the jacket flow through the reactor in cocurrent, and in that the temperature difference of the heat carrier between feed point to the reactor and outlet from the reactor is adjusted to less than 40° C.

The temperature for the cleaving reaction includes all values and subvalues therebetween, especially including 220, 240, 250, 250, 280, 300, 320, 340, 350, 360 and 380° C. The pressure for the cleaving reaction includes all values and subvalues therebetween, especially including 0.2, 0.3, 0.4, 0.5, 0.6, 0.7, 0.8, 0.9, 1.0 and 1.1° C. The temperature drop in the catalyst zone includes all values and subvalues therebetween, especially including 0, 5, 10, 15, 20, 25, 30, 35, 40 and 45° C. The temperature difference of the heat carrier includes all values and subvalues therebetween, especially including 0, 5, 10, 15, 20, 25, 30 and 35° C.

The process according to the invention has the particular advantage that significantly lower catalyst deactivation can be observed. When, for example, MTBE (methyl tert-butyl ether), ETBE (ethyl tert-butyl ether) or TBA (tert-butanol) is cleaved to isobutene and alcohol or water in the gas phase, the reaction is, in order to condense the isobutene formed against cooling water, preferably performed at elevated pressure, for example 0.7 MPa. At this pressure, though, increased deactivation of the catalyst can be observed even at temperatures below 200° C. For the preparation of isoolefins, especially isobutene, cleavage at a temperature in the range of 200° C. to 400° C. is therefore particularly advantageous. One reason for this is possibly that the inventive process can reduce or prevent condensation of relatively high-boiling components on the catalyst, which possibly contributes to catalyst deactivation.

The process according to the invention further has the following advantages: the cleavage is preferably performed in a tube bundle reactor with only one heating circuit. In comparison to reactor systems of more complicated construction, this gives rise to a lower capital investment and lower operating costs. Since the reactor is charged only with one catalyst, a catalyst change can be performed rapidly and inexpensively. Moreover, the catalyst deactivation is reduced or slowed, which prolongs the lifetime of the catalyst. This results in a decrease in the catalyst costs and in the production shutdowns resulting from catalyst change which are caused thereby.

The invention will be described by way of example hereinafter without the invention, the scope of protection which is evident from the claims and the description, being restricted thereto. The claims too form part of the disclosure content of the present invention. When ranges, general formulae or compound classes are specified below, the disclosure shall include not only the appropriate ranges or groups of compounds which are mentioned explicitly but also all subranges and subgroups of compounds which can be obtained by omission of individual values (ranges) or compounds, without these being mentioned explicitly for reasons of better clarity.

In the continuous process according to the invention for preparing isoolefins having 4 to 6 carbon atoms by cleaving compounds of the formula I

R₁—O—R₂ (I)

where R₁=a tertiary alkyl radical having 4 to 6 carbon atoms and R₂=H or an alkyl radical, especially an alkyl radical having 1 to 3 carbon atoms, in the gas phase over a solid catalyst in the temperature range of 200 to 400° C. at a pressure of 0.1 to 1.2 MPa, preferably of 0.5 to 0.9 MPa and more preferably of 0.7 to 0.8 MPa, in a reactor which is equipped with a heating jacket and is heated with a liquid heat carrier, the cleavage is carried out in such a way that the temperature drop in the catalyst zone/reaction zone at any point in relation to the entrance temperature is less than 50° C., in that the reaction mixture in the reactor and the heat carrier in the jacket flow through the reactor in cocurrent, and in that the temperature difference of the heat carrier between feed point to the reactor and outlet from the reactor is adjusted to less than 40° C.

In the process according to the invention, the compounds of the formula I used may, for example, be tertiary alcohols having 4 to 6 carbon atoms. In particular, tert-butanol (TBA) can be cleaved to give isobutene and water. TBA can stem from various industrial processes. One of the most important is the reaction of isobutenic C₄ hydrocarbon mixtures with water. Processes for preparing TBA are described, for example, in DE 103 30 710 and in U.S. Pat. No. 7,002,050. TBA can be used, for example, in pure form, as a TBA/water azeotrope or as another TBA-water mixture.

In the process according to the invention, the compounds of the formula I used may, for example, also be alkyl tert-alkyl ethers. Alkyl tert-alkyl ethers which can be cleaved by the process according to the invention are, for example, MTBE, ETBE or TAME (tert-amyl methyl ether). A process for preparing MTBE is described, for example, in DE 101 02 062. Processes for preparing ETBE are described, for example, in DE 10 2005 062700, DE 10 2005 062722, DE 10 2005 062699 and DE 10 2006 003492.

The compounds of the formula I used in the process according to the invention are preferably tert-butanol, methyl tert-butyl ether, ethyl tert-butyl ether and/or tert-amyl methyl ether. It may be advantageous when a mixture of at least two compounds of the formula I is used. This may be the case, for example, when mixtures of compounds of the formula I are obtained actually in the preparation process of the compound of the formula I to be cleaved owing to the starting substances used. Especially when isobutene is to be obtained by the process according to the invention, it is possible, for example, to use a mixture which comprises tert-butanol and methyl tert-butyl ether. The compounds of the formula I may be fed to the process according to the invention as a pure substance or in a mixture with other compounds. In particular, industrial mixtures which comprise one or more compounds of the formula I can be fed to the process according to the invention.

The mixture comprising MTBE used in the process according to the invention may be MTBE of different quality. The mixture comprising MTBE used may, for example, be pure MTBE, mixtures of MTBE and methanol, industrial MTBE of various qualities or mixtures of industrial MTBE and methanol. In particular, it is possible to use industrial MTBE of various qualities or mixtures of industrial MTBE and methanol. Industrial MTBE (fuel quality) is, especially for economic reasons, the preferred feedstock. Table 1 shows, for example, the typical composition of industrial MTBE from OXENO Olefinchemie GmbH.

TABLE 1
Typical composition of industrial MTBE (fuel quality) from Oxeno
Olefinchemie GmbH.
Parts by mass [kg/kg]

| | |
|---|---|
| 1-Butene/2-Butenes | 0.001000 |
| Pentanes | 0.001500 |
| MTBE | 0.978000 |
| 2-Methoxybutane | 0.003000 |
| Methanol | 0.008500 |
| tert-Butanol | 0.003000 |
| Water | 0.000050 |
| Diisobutene | 0.003300 |

Industrial MTBE can be prepared by known processes by reacting C₄ hydrocarbon mixtures from which the polyunsaturated hydrocarbons have been largely removed, for example raffinate I or selectively hydrogenated crack-C₄, with methanol. A process for preparing MTBE is described, for example, in DE 101 02 062.

In the process according to the invention, it may be particularly advantageous when a stream comprising MTBE is used which is obtained fully or partly by removing low boilers in an optional process step from a stream comprising MTBE.

The removal of low boilers may be advantageous especially when the stream containing MTBE comprises, for example, C₄ and/or C₅ hydrocarbons. The low boilers, for example C₄ and/or C₅ hydrocarbons, can preferably be removed from the stream in the optional process step in a distillation column. The distillation column is preferably operated in such a way that the low boilers can be removed as the top product.

The removal of the low boilers is preferably performed in a distillation column which has 30 to 75 theoretical plates, preferably 40 to 65 theoretical plates and more preferably 40 to 55 theoretical plates. Depending on the number of stages realised, the composition of the MTBE used and the purity of C₄ and C₅ hydrocarbons required, the column is preferably operated with a reflux ratio from 150 to 350, in particular from 200 to 300. The reflux ratio includes all values and subvalues therebetween, especially including 160, 180, 200, 220, 240, 250, 260, 280, 300, 320 and 340. The column in the optional process step is preferably operated at an operating pressure of 0.2 to 0.6 MPa_{(abs)}, preferably of 0.3 to 0.4 MPa_{(abs)}. To heat the column, for example, 0.4 MPa steam can be used. Depending on the operating pressure selected, the condensation can be effected against cooling brine, cooling water or air. The top vapours of the column can be condensed fully or only partly, so that the top product can be drawn off either in liquid or vapour form. The top product can be utilized thermally or be utilized as a feedstock of a synthesis gas plant. The bottom product can be sent directly to the cleavage.

The process according to the invention is performed in such a way that the temperature does not fall below 200° C. at any point in the catalyst zone. The entrance temperature of the gaseous reactant is therefore preferably above 200° C., preferably significantly above 200° C. The entrance temperature of the reactant can be established in a heater connected upstream of the reactor. When the reactant used is a reactant comprising MTBE as the compound of the formula I, the entrance temperature is preferably at least 230° C., preferably greater than 250° C.

When fresh catalyst is used, especially when fresh magnesium oxide/aluminium oxide/silicon oxide catalyst is used, in the MTBE cleavage, the entrance temperature is preferably from 250 to 270° C. In the course of operation, it may be advantageous to raise the entrance temperature up to 400° C. to keep the conversion constant with increasing deactivation of the catalyst. When the conversion can no longer be maintained on attainment of 400° C., it may be advantageous to replace the catalyst fully or partly.

The temperature drop in the catalyst zone at any point in relation to the entrance temperature is less than 50° C., preferably less than 40° C. and more preferably 1 to 30° C. The maximum pressure drop can be adjusted by numerous parameters, for example by the temperature of the heat carrier used for heating and by the rate with which the heat carrier flows through the jacket.

The reactor is preferably operated in straight pass with a weight hourly space velocity (WHSV) in kilograms of reactant per kilogram of catalyst per hour) of 0.1 to 5 h⁻¹, in particular of 1 to 3 h⁻¹. The weight hourly space velocity includes all values and subvalues therebetween, especially including 0.5, 1, 1.5, 2, 2.5, 3, 3.5, 4, and 4.5 h⁻¹.

The reactor may be arranged in any spatial direction. When the reactor has reactor tubes, these may likewise point in any spatial direction. However, the reactor is preferably configured such that the reactor and the reactor tubes are aligned vertically. In a vertical reactor, the heat carrier is preferably supplied at the highest point or close to the highest point in the jacket and drawn off at the lowest point or close to the lowest point of the reactor, or vice versa. The reaction mixture in the reaction zone and the heat carrier in the jacket flow through the reactor in the same direction. More preferably, the heat carrier and the reaction mixture flow, respectively, through the jacket of the reactor and the reaction zone of the reactor from the top downwards.

In order to achieve more uniform heating of the reaction zone, it may be advantageous to feed the heat carrier into the reactor not just at one point but at several points at about the same height. In order to avoid a relatively large temperature drop in the middle tubes in comparison to edge tubes when a tube bundle reactor is used, it may be advantageous to provide nozzles for the heat carrier in the feed or in the feeds, which promote the transport of the heat carrier to the middle tubes. In this way, it is possible to avoid temperature variations over the cross section of the tube bundle.

The heat carrier can leave the reactor at one or more point(s). When the reactor is flowed through by the heat carrier from the top downwards, it should be ensured by construction measures that there is full flow around the reaction zones, for example the reaction tubes, with heat carrier.

The heat carrier may be brought to the desired temperature by direct or indirect heating outside the reactor and be pumped through the reactor.

The heat carriers used may be salt melts, water or heat carrier oils. For the temperature range of 200 to 400° C., the use of heat carrier oils is advantageous, since heating circuits comprising them entail a smaller capital investment in comparison to other technical solutions. Heat carrier oils which can be used are, for example, those which are sold under the tradenames Marlotherm (e.g. Marlotherm SH from Sasol Olefins & Surfactants GmbH), Diphyl (from Bayer), Dowtherm (from Dow) or Therminol (from Therminol). These synthetic heat carrier oils are based essentially on thermally stable ring hydrocarbons.

The heat carrier is preferably passed into the heating jacket of the reactor at a temperature which is 10 to 40° C., preferably 10 to 30° C., higher than the temperature of the reactant flowing into the reactor. The temperature difference of the liquid heat carrier over the reactor, i.e. between entrance temperature of the heat carrier on entry into the heating jacket and the exit temperature of the heat carrier on exit from the heating jacket is preferably less than 40° C., preferentially less than 30° C. and more preferably 10 to 25° C. The temperature difference can be adjusted by the mass flow of the heat carrier per unit time (kilograms per hour) through the heating jacket.

In the process according to the invention, it is possible to use all solid catalysts which enable the cleavage of tert-alcohols and alkyl tert-alkyl ethers to isoolefins in the temperature range of 200 to 400° C. Preference is given to using catalysts with which a reaction rate of at most 400 mol/(h·kg_{CAT}), preferably from 1 to 400 mol/(h·kg_{CAT}), is achieved in the reactor. The reaction rate includes all values and subvalues therebetween, especially including 5, 10, 15, 20, 40, 60, 80, 100, 120, 140, 160, 180, 200, 220, 240, 260, 280, 300, 320, 340, 360 and 380 mol/(h·kg_{CAT}).

The catalysts used in the process according to the invention may, for example, be metal oxides, mixed metal oxides, especially those which contain silicon oxide and/or aluminium oxide, acids on metal oxide supports or metal salts or mixtures thereof.

In the process according to the invention, MTBE is cleaved to isobutene and methanol in the gas phase preferably by using catalysts which consist in a formal sense of magnesium oxide, aluminium oxide and silicon oxide. Such catalysts are described, for example, in U.S. Pat. No. 5,171,920 in Example 4 and in EP 0 589 557.

Particular preference is given to using catalysts which, in a formal sense, comprise magnesium oxide, aluminium oxide and silicon oxide, and which have a proportion of magnesium oxide of 0.5 to 20% by mass, preferably of 5 to 15% by mass and more preferably of 10 to 15 by mass, a proportion of aluminium oxide of 4 to 30% by mass, preferably of 10 to 20% by mass and a proportion of silicon dioxide of 60 to 95% masss, preferably of 70 to 90% by mass. The amount of magnesium oxide includes all values and subvalues therebetween, especially including 1, 2, 4, 6, 8, 10, 12, 14, 16, 18% by mass based on the weight of the catalyst. The amount of aluminum oxide includes all values and subvalues therebetween, especially including 5, 10, 15, 20, 25, 30 and 35% by mass based on the weight of the catalyst. The amount of silicon dioxide includes all values and subvalues therebetween, especially including 65, 70, 75, 80, 85, 90, 95% by mass based on the weight of the catalyst.

It may be advantageous when the catalyst, in addition to the magnesium oxide, comprises an alkali metal oxide. This may, for example, be selected from Na₂O or K₂O. The catalyst preferably comprises Na₂O as the alkali metal oxide. The catalyst used with preference preferably has a BET surface area (determined by volumetric means with nitrogen to DIN ISO 9277) of 200 to 450 m²/g, preferably of 200 to 350 m²/g. The BET surface area includes all values and subvalues therebetween, especially including 220, 240, 260, 280, 300, 320, 340, 360, 380, 400, 420 and 440 m²/g. When the inventive catalyst is applied as an active composition on a support, only the active composition has a BET surface area in the range specified. The material containing catalyst and support may, in contrast, depending on the properties of the support, have a significantly different BET surface area, especially a smaller BET surface area.

The pore volume of the catalyst is preferably 0.5 to 1.3 ml/g, preferentially 0.65 to 1.1 ml/g. The pore volume of the catalyst includes all values and subvalues therebetween, especially including 0.6, 0.7, 0.8, 0.9, 1, 1.1, 1.2 ml/g. The pore volume is preferably determined by the cyclohexane method. In this method, the sample to be tested is first dried at 110° C. to constant weight. Subsequently, approx. 50 ml of the sample weighed accurately to 0.01 g are introduced into a cleaned impregnation tube dried to constant weight, which has an outlet orifice with a ground-glass tap at the lower end. The outlet orifice is covered with a small piece of polyethylene, which prevents blockage of the outlet orifice by the sample. After the impregnation tube has been filled with the sample, the tube is carefully sealed air-tight. Subsequently, the impregnation tube is connected to a water-jet pump, the ground-glass tap is opened and the water jet is used to establish a vacuum in the impregnation tube of 20 mbar. The vacuum can be checked on a parallel vacuum meter. After 20 min, the ground-glass tap is opened and the evacuated impregnation tube is subsequently connected to a cyclohexane receiver in which an accurately measured volume of cyclohexane is initially charged, such that opening of the ground-glass tap results in suction of cyclohexane from the receiver into the impregnation tube. The ground-glass tap remains open until the entire sample has been flooded with cyclohexane. Subsequently, the ground-glass tap is closed again. After 15 min, the impregnation tube is aerated cautiously and the unabsorbed cyclohexane is discharged into the receiver. Cyclohexane adhering in the impregnation tube or in the outlet orifice or the connection to the cyclohexane receiver can be conveyed via the aeration line into the receiver by a single cautious pressure impulse from a suction ball. The volume of the cyclohexane present in the receiver is noted. The pore volume is determined from the absorbed volume of cyclohexane, which is determined from the cyclohexane volume in the receiver before the measurement minus the cyclohexane volume in the receiver after the measurement, divided by the mass of the sample analysed.

The mean pore diameter (preferably determined on the basis of DIN 66133) of the catalyst is preferably 5 to 20 nm, preferably 8 to 15 nm. More preferably, at least 50%, preferably over 70%, of the total pore volume (sum of the pore volume of the pores having a pore diameter of greater than or equal to 3.5 nm determined by mercury porosimetry to DIN 66133) of the catalyst is accounted for by pores having a diameter of 3.5 to 50 nm (mesopores).

In the process according to the invention, preference is given to using catalysts which have a mean particle size (determined by screen analysis) of 10 μm to 10 mm, preferably 0.5 mm to 10 mm, more preferably a mean particle size of 1 to 5 mm. Preference is given to using solid catalysts which have a mean particle size d₅₀, of 2 to 4 mm, in particular of 3 to 4 mm. The mean particle size includes all values and subvalues therebetween, especially including 50, 100, 200, 300, 400, 500, 600, 700, 800, 900, 1000, 1500, 2000, 2500, 3000, 3500, 4000, 4500, 5000, 5500, 6000, 6500, 7000, 7500, 8000, 8500, 9000, and 9500 μm.

In the process according to the invention, the catalyst may be used in the form of shaped bodies. The shaped bodies may assume any shape. Preference is given to using the catalyst in the form of shaped bodies in the form of spheres, extrudates or tablets. The shaped bodies preferably have the abovementioned mean particle sizes.

The catalyst may also be applied on a support, for example a metal, plastic or ceramic support, preferably on a support which is inert in relation to the reaction in which the catalyst is to be used. In particular, the inventive catalyst may be applied to a metal support, for example a metal plate or a metal fabric. Such supports provided with the inventive catalyst may be used, for example, as internals in reactors or reactive distillation columns. The supports may also be metal, glass or ceramic spheres or spheres of inorganic oxides. When the inventive catalyst is applied on an inert support, the mass and composition of the inert support are not taken into account in the determination of the composition of the catalyst.

The catalysts which comprise, in a formal sense, magnesium oxide (MgO), aluminium oxide (Al₂O₃) and silicon dioxide (SiO₂) and are to be used with particular preference may be prepared, for example, by a process which comprises the steps of

a) treating an aluminosilicate with an acidic aqueous magnesium salt solution and

b) calcining the aluminosilicate treated with aqueous magnesium salt solution.

Aluminosilicates shall be understood to mean compounds which are essentially containing, in a formal sense, fractions of aluminium oxide (Al₂O₃) and silicon dioxide (SiO₂). The aluminosilicates may also contain small fractions of alkali metal oxides or alkaline earth metal oxides. In the process, the aluminosilicates used may also be zeolites, for example zeolite A, X, Y, USY or ZSM-5, or amorphous zeolites (for example MCM 41 from Mobil Oil). The aluminosilicates used in the process may be amorphous or crystalline. Suitable commercial aluminosilicates which can be used as starting materials in the process according to the invention are, for example, aluminosilicates which have been prepared by precipitation, gelation or pyrolysis. In the process, preference is given to using aluminosilicates with 5 to 40% by mass, preferably 10 to 35% by mass, of aluminium oxide, and 60 to 95% by mass, preferably 65 to 90% by mass, of silicon dioxide (based on the dry mass; treatment: calcination at 850° C. for 1 h). The amount of aluminium oxide includes all values and subvalues therebetween, especially including 10, 15, 20, 25, 30, 35% by mass based on the weight of the aluminosilicate. The amount of silicon dioxide includes all values and subvalues therebetween, especially including 65, 70, 75, 80, 85 and 90% by mass. The composition of the aluminosilicates used and of the catalysts obtained can be determined, for example, by classical analysis, fusion with borax and XFA. (X-ray fluorescence analysis), energy-dispersive X-ray analysis, flame spectroscopy (Al and Mg, not Si), wet digestion and subsequent ICP-OES (optical emission spectrometry with inductively coupled high-frequency plasma) or atomic absorption spectroscopy. A particularly preferred aluminosilicate which can be used in the process has a formal proportion of Al₂O₃ of 13% by mass and a proprotion of silicon dioxide of 76% by mass. Such an aluminosilicate is supplied by Grace Davison under the name Davicat O 701.

The aluminosilicate can be used in the process in a wide variety of different forms. For instance, the aluminosilicate can be used in the form of shaped bodies, for example tablets, pellets, granules, strands or extrudates. The aluminosilicate can also be used in the form of aluminosilicate powder. The starting material used may be powders with different mean particle size and different particle size distribution. In the process according to the invention, preference is given to using an aluminosilicate powder in which 95% of the particles have a mean particle size of 5 to 100 μm, preferably 10 to 30 μm and more preferably 20 to 30 μm. The particle size can be determined, for example, by laser diffraction with a particle analyser from Malvern, for example the Mastersizer 2000.

To prepare the aqueous magnesium salt solution, magnesium compounds which are water-soluble or are converted to water-soluble compounds by adding an acid are used. The salts used are preferably the nitrates. Preference is given to using magnesium salt solutions which, as magnesium salts, comprise the salts of strong mineral acids, for example magnesium nitrate hexahydrate or magnesium sulphate heptahydrate. The acidic aqueous alkali metal or alkaline earth metal salt solution used preferably has a pH of less than 6, preferably of less than 6 to 3 and more preferably of 5.5 to 3.5. The pH can be determined, for example, with the aid of a glass electrode or indicator paper. When the salt solution has a pH which is greater than or equal to 6, the pH can be adjusted by adding an acid, preferably the acid whose alkali metal and/or alkaline earth metal salt is present in the solution. When the salts present in the alkali metal and/or alkaline earth metal salt solution are the nitrates, the acid used is preferably nitric acid. The magnesium content of the magnesium salt solution used is preferably 0.1 to 3 mol/l, preferably 0.5 to 2.5 mol/l.

The treatment in step a) can be effected in various ways which are suitable for contacting the aluminosilicate with the magnesium salt solution. Possible treatment methods are, for example, impregnation, saturation, spraying or immersing the aluminosilicate with the magnesium salt solution. It may be advantageous when the treatment of the aluminosilicate is effected in such a way that the magnesium salt solution can act on the aluminosilicate for at least 0.1 to 5 h, preferably 0.5 to 2 h. Such an action time may be advantageous especially when the treatment is effected by simple saturation.

In a preferred embodiment of the inventive step a) of the process according to the invention, the treatment of aluminosilicate, especially aluminosilicate shaped bodies, with the magnesium salt solution can be effected, for example, by vacuum impregnation in a vacuum impregnation unit suitable therefor. In this type of treatment, the aluminosilicate in the vacuum imprengation unit is first evacuated. Subsequently, the magnesium salt solution is sucked in up to above the upper edge of the support bed, so that the entire aluminosilicate is covered with the solution. After an action time which is preferably 0.1 to 10 h, preferentially 0.5 to 2 h, the solution which has not been taken up by the support is discharged.

In a further preferred embodiment of the inventive step a) of the process according to the invention, the treatment of aluminosilicate, especially aluminosilicate shaped bodies, with the alkali metal and/or alkaline earth metal solution can be effected, for example, by spraying or immersing the aluminosilicate. The spraying or immersion of the aluminosilicate with the magnesium salt solution is preferably effected by spraying or pouring the solution onto the aluminosilicate rotating in a drum. The treatment can be effected in one step, i.e. the entire amount of magnesium salt solution is added at the start to the aluminosilicate in one step. However, the salt solution can also be metered in by spraying or immersion in small portions, the period of addition being preferably 0.1 to 10 h and preferentially 1 to 3 h. The amount of salt solution is preferably such that the entire solution of the aluminosilicate is taken up. Saturation in particular, but also spraying or immersion, can be performed in customary industrial apparatus, for example conical mixers or intensive mixers, as supplied, for example, by Eirich.

The treatment of the aluminosilicate with the magnesium salt solution in step a) can be effected in one step or in a plurality of partial steps. In particular, it is possible to perform the treatment in two or more partial steps. In each of the individual partial steps, the same magnesium salt solution can be used in each case, or else a magnesium salt solution of different concentration can be used in each partial step. For example, initially only a portion of the magnesium salt solution can be added to the aluminosilicate and, optionally after intermediate drying, the remaining amount of the magnesium salt solution used can be added at the same temperature or a different temperature. It is not only possible that step a) is performed in two or more partial steps. It is likewise possible that the process has a plurality of steps a). In this case too, magnesium salt solutions of the same concentration or different concentrations can be used in the different steps a).

The treatment in step a) can be performed preferably at a temperature of 10 to 120° C., preferentially of 10 to 90° C., more preferably of 15 to 60° C. and most preferably at a temperature of 20 to 40° C.

It may be advantageous when one or more additives is/are added or admixed to the aluminosilicate or to the magnesium salt solution in step a). Such additives may, for example, be binders, lubricants or shaping assistants. A suitable binder may, for example, be boehmite or pseudoboehmite, as supplied, for example, under the name Disperal (boehmite having a formal Al₂O₃ content of approx. 77% by mass) by Sasol Deutschland GmbH. When boehmite, especially Disperal, is added as a binder, it is preferably added as a gel which can be obtained, for example, by stirring 197 parts by mass of Disperal into 803 parts by mass of 1.28% by mass aqueous nitric acid, stirring thoroughly at 60° C. for 3 h, cooling to room temperature and replacing any evaporated water. The shaping assistants used may, for example, be silicas, especially pyrogenic silicas, as sold, for example, by Degussai AG under the name Aerosil, bentonites, clays, kaolin, kaolinite, ball clay and other substances familiar for this purpose to those skilled in the art. The lubricants added, whose use may be advantageous for improved tableting, may, for example, be graphite.

One or more of the abovementioned additives may be added in step a) in various ways. In particular, the addition can be effected during the treatment of the aluminosilicate with the magnesium salt solution. For example, aluminosilicate, additive and magnesium salt solution can be charged into an industrial apparatus and then mixed intimately. Another possibility is to first mix the aluminosilicate with the additive and then to add the magnesium salt solution. In a further variant, additive and magnesium salt solution can be metered simultaneously to the aluminosilicate. The addition can be effected in each case in one batch, in portions or by spraying. The addition time is preferably less than 5 h, preferentially less than 3 h. It may be advantageous to continue to mix the mixture for 0.1 to 10 h, preferably for 0.5 to 3 h.

The preparation process for the catalyst used with preference has at least one process step b) in which the aluminosilicate treated with alkali metal and/or alkaline earth metal salt solution is calcined. The calcination is effected preferably in a gas stream, for example in a gas stream which comprises, for example, air, nitrogen, carbon dioxide and/or one or more noble gases, or consists of one or more of these components. Preference is given to effecting the calcining using air as the gas stream.

The calcination in process step b) is performed preferably at a temperature of 200 to 1000° C., preferably of 300 to 800° C. The calcination is effected preferably for a time of 0.1 to 10 hours, preferably 1 to 5 hours. Particular preference is given to performing the calcining at a temperature of 200 to 1000° C., preferably 300 to 800° C., for 0.1 to 10 hours, preferably 1 to 5 hours.

The industrial calcination can preferably be performed in a shaft oven. However, the calcination can also be performed in other known industrial apparatus, for example fluidized bed calciners, rotary tube ovens or tray ovens.

It may be advantageous when a step c) in which the aluminosilicate treated with magnesium salt solution is dried is performed between steps a) and b). The drying in step c) can be effected at a temperature of 100 to 140° C. The drying is preferably effected in a gas stream. The drying can be performed, for example, in a gas stream which comprises, for example, air, nitrogen, carbon dioxide and/or one or more noble gases, or consists of one or more of these components. The intermediate drying step after the treatment with alkali metal and/or alkaline earth metal salt solution and before the calcining can achieve the effect that no large amounts of steam are released in the course of calcination. In addition, the drying can prevent water which evaporates spontaneously in the course of calcining from destroying the shape of the catalyst.

Depending on the desired shape in which the catalyst is to be present, it may be advantageous to adjust the preparation process appropriately by additional process steps. When, for example, pulverulent catalyst is to be prepared by the process, the aluminosilicate can be used in the form of aluminosilicate powder and, for example, treated with the magnesium salt solution (for example by impregnation), for example in a conical mixer, optionally dried and then calcined. However, a pulverulent catalyst can also be prepared by processing a shaped catalyst body to give a pulverulent catalyst by grinding and screening.

The shaped catalyst bodies may be present, for example, in the form of extrudates, spheres, pellets or tablets. In order to arrive at the shaped catalyst (shaped catalyst bodies), depending on the particular shaping variant, it is possible to perform further process steps, for example shaping, grinding or screening, in addition to the process steps of treatment, drying, calcination. Shaping assistants can be introduced at various points in the process. The shaped catalyst bodies can be prepared in various ways:

In a first variant, shaped catalyst bodies, especially inventive shaped catalyst bodies, can be obtained by treating shaped aluminosilicate bodies with an acidic aqueous magnesium salt solution, optionally drying and then calcining.

In a second embodiment, a shaped catalyst body can be obtained by first treating an aluminosilicate powder with an acidic aqueous magnesium salt solution, then optionally drying and subsequently calcining it, and subsequently processing the resulting catalyst powder by processes customary in industry, for example compaction, extrusion, pelletization, tabletting, granulation or coating to give shaped catalyst bodies. Additives required for the shaping, for example binders or further assistants, can be added at various points in the preparation process, for example in process step a). When a shaped body is prepared from an aluminosilicate powder as a starting material, it is possible to start from powders with different mean particle size and different particle size distribution. For the preparation of shaped bodies, preference is given to using an aluminosilicate powder in which 95% of the particles have a particle size of 5 to 100 μm, preferably 10 to 30 μm and more preferably 20 to 30 μm (determined by laser diffraction; see above).

In a third embodiment of the process, pellets of the catalyst can be obtained by, in process step a), treating an aluminosilicate powder with an acidic aqueous magnesium salt solution, optionally drying (process step c)) and then calcining in process b), and pelletizing the catalyst powder thus obtained with addition of binder, for example in an Eirich mixer, and drying the resulting pellets in a further process step c) and then calcining them in a further process step b).

In a fourth embodiment of the preparation process, pellets of the catalyst can be obtained by, in process step a), mixing an aluminosilicate powder, binder and acidic aqueous magnesium salt solution, and pelletizing the aluminosilicate powder thus treated, for example in an Eirich mixer, and drying the resulting moist pellets in process step c) and then calcining them in a gas stream in process step b).

In a fifth embodiment of the preparation process, tablets of the catalyst can be obtained by, in process step a), mixing an aluminosilicate powder, binder, optionally lubricant and acidic aqueous magnesium salt solution, and pelletizing the aluminosilicate powder thus treated, for example in an Eirich mixer, to give micropellets, preferably having a mean diameter of 0.5 to 10 mm, preferably 1 to 5 mm and more preferably of 1 to 3 mm (the particle size can be determined, for example, by screen analysis), and drying the resulting moist pellets in process step c) and then optionally calcining them in a gas stream in process step b). The resulting pellets may then, unless already done in process step a), be mixed with a lubricant, for example graphite, and then tabletted on a commercial tabletting press, for example a rotary tabletting press. The tablets may then, if process step b) is yet to be performed, be calcined in process step b), or optionally post-calcined.

In a sixth embodiment of the preparation process, tablets of the catalyst can be obtained by grinding preshaped shaped catalyst bodies, as can be obtained, for example, as pellets in embodiment three or four, and screening the granule/powder obtained, so as to obtain a tablettable granule of catalyst, and adding lubricants to this granule. The granule thus prepared can then be tabletted. The tablets may then, if process step b) is yet to be performed, be calcined in process step b). The addition of a lubricant can be dispensed with when a lubricant has already been added in the course of preparation of the pellets, for example in process step a).

In a seventh embodiment of the process according to the invention, materials/supports coated with the catalyst can be prepared. In this embodiment, a catalyst powder is first prepared by, in process a), treating an aluminosilicate powder with an acidic aqueous magnesium salt solution, optionally drying (process step c)) and optionally calcining (process step b)). The catalyst powder thus obtained is then suspended in a suspension medium, for example water or alcohol, for which a binder can optionally be added to the suspension. The suspension thus prepared can then be applied to any material. The application is followed by optional drying (process step c)) and then calcining (process step b)). In this way, materials/supports coated with the preferred catalyst can be provided. Such materials/supports may, for example, be metal plates or fabric, as can be used as internals in reactors or columns, especially reactive distillation columns, or else metal, glass or ceramic spheres, or spheres of inorganic oxides.

In an eighth embodiment of the preparation process, extrudates of the catalyst, especially of the inventive catalyst, can be obtained by, in process step a), mixing an aluminosilicate powder, acidic aqueous alkali metal and/or alkali metal salt solution, binder, for example Disperal, and further shaping assistants customary for extrusion, for example clays such as bentonite or attapulgite, in a kneader or Eirich mixer, and extruding them in an extruder to give extrudates, preferably having a mean diameter of 0.5 to 10 mm, preferentially of 1 to 5 mm and more preferably of 1 to 3 mm, and drying the resulting moist extrudates optionally in process step c) and then calcining them in a gas stream in process step b).

The process according to the invention for preparing isoolefins, especially isobutene, by cleavage of tertiary alcohols or alkyl tert-alkyl ethers in the gas phase over a solid catalyst can be performed in all suitable reactors which have a reaction zone (catalyst zone) which comprises the catalyst and is spatially separate from a heating jacket through which the heat carrier flows. The process according to the invention is preferably performed in a plate reactor, in a tubular reactor, in a plurality of tubular reactors or plate reactors connected in parallel, or in a tube bundle reactor. Preference is given to performing the process according to the invention in a tube bundle reactor.

It is pointed out that the hollow bodies in which the catalyst is disposed need not only be tubes in the customary sense of the term. The hollow bodies may also have noncircular cross sections. They may, for example, be elliptical or triangular.

The materials used for the construction of the reactor, especially the material which divides the reaction zone from the heating jacket, preferably has a high coefficient of thermal conductivity (greater than 40 W/(m·.K)). The material used which has a high coefficient of thermal conductivity is preferably iron or an iron alloy, for example steel.

When the process according to the invention is performed in a tube bundle reactor, the individual tubes preferably have a length of 1 to 15 m, preferably of 3 to 9 m and more preferably 5 to 9 m. The individual tubes in a tube bundle reactor used in the process according to the invention preferably have an internal diameter of 10 to 60 mm, preferably of 20 to 40 mm and more preferably of 24 to 35 mm. It may be advantageous when the individual tubes of the tube bundle reactor used in the process according to the invention have a thickness of the tube wall of 1 to 4 mm, preferably of 1.5 to 3 mm.

In a tube bundle reactor used in the process according to the invention, the tubes are preferably arranged in parallel. The tubes are preferably arranged uniformly. The arrangement of the tubes may, for example, be square, triangular or rhombus-shaped. Particular preference is given to an arrangement in which the centres of three mutually adjacent tubes connected virtually form an equilateral triangle, i.e. the tubes have the same separation. The process according to the invention is preferably performed in a tube bundle reactor in which the tubes have a separation from one another of 3 to 15 mm, more preferably of 4 to 7 mm.

The process according to the invention is preferably operated in such a way that the conversion of compounds of the formula I is in the range of 70 to 98%, in particular in the range of 90 to 95%.

The present invention relates in particular to the preparation of isobutene and methanol by cleaving MTBE. The reactor operated in accordance with the invention may be an integral part of processes for preparing isobutene from industrial MTBE. Such processes have already been described frequently in the background art.

The cleavage mixtures can be worked up in a known manner, for example as described in the related art. A preferred type of workup is described below.

In order to work up the cleavage product mixture, the cleavage product mixture can be separated in a first distillation step into a top stream comprising isoolefin and a bottom stream comprising unconverted compound of the formula I. The distillative separation of the cleavage product into a top stream comprising isoolefin and a bottom stream comprising unconverted compound of the formula I is effected in at least one column, preferably in exactly one distillation column.

A distillation column used with preference in the distillative separation has preferably 20 to 55 theoretical plates, preferably 25 to 45 theoretical plates and more preferably 30 to 40 theoretical plates. Depending on the number of stages realised, the composition of the reactor effluent and the required purities of distillate and bottom product, the reflux ratio is preferably less than 5, preferentially less than 1. The operating pressure of the column may preferably be adjusted to from 0.1 to 2.0 MPa_{(abs)}. In order to save one compressor, it may be advantageous to operate the column at a lower pressure than the pressure with which the cleavage reactor is operated. In order to condense isobutene against cooling water, a pressure of approx. 0.5 MPa_{(abs)} is necessary. When the cleavage is operated, for example, at a pressure of 0.65 MPa_{(abs)}, it may be advantageous when the distillation is performed at an operating pressure of from 0.55 to 0.6 MPa_{(abs)}. To heat the evaporator, for example, 0.4 MPa steam may be used. The bottom product preferably contains unconverted compound of the formula I, alcohol or water and optionally by-products, for example diisobutene and/or 2-methoxybutane. The top product is preferably isoolefin, especially isobutene having a purity greater than 95% by mass, based on the overall top product.

Optionally, the workup of the cleavage product mixture can be performed in at least one column designed as a reactive distillation column. This embodiment of the process according to the invention has the advantage that the conversion of compound of the formula I in the entire process can be increased by cleaving a portion of the compound of the formula I unconverted in the cleavage in the reaction part of the reactive distillation column to give isoolefin and alcohol or water.

The catalysts used in the reaction part of the reactive distillation column may be all catalysts which are suitable for the cleavage of compounds of the formula I. The catalysts used are preferably acidic catalysts. A particularly preferred group of acidic catalysts for use in the reaction part of the reactive distillation column is that of solid acidic ion exchange resins, especially those having sulphonic acid groups. Suitable acidic ion exchange resins are, for example, those which are prepared by sulphonating phenol/aldehyde condensates or cooligomers of aromatic vinyl compounds. Examples of aromatic vinyl compounds for preparing the cooligomers are: styrene, vinyltoluene, vinylnaphthalene, vinylethylbenzene, methylstyrene, vinylchlorobenzene, vinylxylene and divinylbenzene. In particular, the cooligomers which are formed by reacting styrene with divinylbenzene are used as a precursor for the preparation of ion exchange resins with sulphonic acid groups. The resins may be prepared in gel form, macroporous form or sponge form. The properties of these resins, especially specific surface area, porosity, stability, swelling and shrinkage, and exchange capacity, can be varied by the preparation process.

In the reaction part of the reactive distillation column, the ion exchange resins are used in their H form or at least partly in the H form. Strongly acidic resins of the styrene-divinylbenzene type are sold, inter alia, under the following trade names: Duolite C20, Duolite C26, Amberlyst 15, Amberlyst 35, Amberlyst 46, Amberlite IR-120, Amberlite 200, Dowex 50, Lewatit SPC 118, Lewatit SPC 108, K2611, K2621, OC 1501.

The pore volume of the ion exchange resins used is preferably 0.3 to 0.9 ml/g, in particular 0.5 to 0.9 ml/g. The particle size of the resin is preferably 0.3 mm to 1.5 mm, preferentially 0.5 mm to 1.0 mm. The particle size distribution selected can be narrow or wide. For example, ion exchange resins with very homogeneous particle size (monodisperse resins) can be used. The capacity of the ion exchanger is, based on the supply form, preferably 0.7 to 2.0 eq/l, in particular 1.1 to 2.0 eq/l.

In the reaction part of a column optionally designed as a reactive distillation column, the catalyst may either be integrated in the packing, for example in KataMax® (as described in EP 0 428 265) or KataPak® (as described in EP 0 396 650 or DE 298 07 007.3 U1), or polymerized onto shaped bodies (as described in U.S. Pat. No. 5,244,929).

The reactive distillation column preferably has, above the catalyst packing, a region of purely distillative separation. The zone above the catalyst packing has preferably 5 to 25, in particular 5 to 15 theoretical plates. The separating zone below the catalyst comprises preferably 5 to 35, preferentially 5 to 25 theoretical plates. The feed to the reactive distillation column may be above or below, preferably above, the catalyst zone.

The compound of the formula I is converted to isoolefin and alcohol or water in the reactive distillation preferably within a temperature range of from 60 to 140° C., preferably from 80 to 130° C., more preferably from 90 to 110° C. (temperature in the region of the column in which the catalyst is disposed; the bottom temperature can be significantly higher).

For the operating pressure of the reactive distillation column, similar operating conditions to those for the above-described embodiment as a pure distillation column can in principle be selected. Thus, preference is given to setting an operating pressure of the reactive distillation column of from 0.1 to 1.2 MPa_{(abs)}. In order to save one compressor, it may be advantageous to operate the column at a lower pressure than the pressure with which the cleavage reactor is operated. In order to be able to condense isobutene against cooling water, a pressure of approx. 0.5 MPa_{(abs)} is necessary. When the cleavage is operated, for example, at a pressure of 0.65 MPa_{(abs)}, it may be advantageous when the reactive distillation is performed with an operating pressure of 0.55 to 0.6 MPa_{(abs)}. To heat the evaporator, for example, 0.4 MPa steam may be used.

The hydraulic loading in the catalytic packing of the column is preferably 10 to 110%, preferentially 20 to 70%, of its flood point loading. Hydraulic stress on a distillation column is understood to mean the uniform flow demand on the column cross section by the ascending vapour mass stream and the refluxing liquid mass stream. The upper loading limit indicates the maximum loading by vapour and reflux liquid, above which the separating action falls owing to entrainment or accumulation of the reflux liquid as a result of the ascending vapour stream. The lower loading limit indicates the minimum load below which the separating action falls or collapses owing to irregular flow or to the column running empty—for example of the trays (Vauck/Müller, “Grundoperationen chemischer Verfahrenstechnik” [Basic operations in chemical process technology], p. 626, VEB Deutscher Verlag für Grundstoffindustrie).

In the case of an embodiment of the column as a reactive distillation column too, preference is given to obtaining a bottom product which comprises unconverted compound of the formula I and alcohol or water and, if appropriate, by-products, for example diisoolefins.

The top product preferably comprises isoolefin with a purity greater than 95% by mass.

The top product which is obtained in this distillation and which preferably consists of isoolefin to an extent of greater than 95% by mass can be used directly as a saleable product or be purified further.

Since isobutene forms a minimum azeotrope with methanol, the top product obtained in the first distillation step, in addition to the main isobutene product, contains methanol in particular. Further components which may be present in the top product are, for example dimethyl ether, which may have been formed, for example, by condensation of methanol, and linear butenes (1-butene, cis-2-butene, trans-2-butene), which may have been formed, for example, by decomposition of 2-methoxybutane, and water.

A portion of the dimethyl ether can optionally be removed from the top product actually within the distillation step by operating the condenser on the distillation column or reactive distillation column as a partial condenser. The fraction present in the top product can be condensed therein and a portion of the dimethyl ether present in the top product can be drawn off in gaseous form.

Marketable isoolefin qualities, especially isobutene qualities, are typically virtually free of alcohol, especially methanol. Methanol can be removed from the top product obtained in the first distillation step by processes known per se, for example by extraction. The extraction of methanol from the top product can be performed, for example, with water or an aqueous solution as an extractant, for example in an extraction column. The extraction is preferably performed with water or an aqueous solution in an extraction column which preferably has 4 to 16 theoretical plates. The extractant is preferably conducted through the extraction column in countercurrent in relation to the stream to be extracted. The extraction is preferably performed at a temperature of 15 to 50° C., preferably 25 to 40° C. For example, in the case of use of an extraction column having more than 6 theoretical plates, which is operated at a pressure of 0.9 MPa_{(abs)} and a temperature of 40° C., it is possible to obtain a water-saturated isoolefin, especially isobutene having an isoolefin content or isobutene content of over 99% by mass.

The methanolic water extract obtained in the extraction can be separated by distillation into water and methanol. The water can be recycled into the extraction stage as an extractant. The methanol can be utilized for customary industrial syntheses, for example esterifications or etherifications.

The moist isoolefin stream or isobutene stream from the extraction column can be worked up to dry isoolefin or isobutene in a further distillation column by removing water and optionally dimethyl ether. The dry isoolefin or isobutene is obtained as the bottom product. In the condensation system at the top of the column, after a phase separation, water can be drawn off in liquid form and dimethyl ether in gaseous form. A distillation column used with preference for the drying has preferably 30 to 80 theoretical plates, preferably 40 to 65 theoretical plates. The reflux ratio is, depending on the number of stages realised and the required purity of the isoolefin or isobutene, preferably less than 60, preferentially less than 40. The operating pressure of this distillation column used for the drying may preferably be set between 0.1 and 2.0 MPa_{(abs)}.

Workup of isobutene by extraction and distillation is described in detail, for example, in DE 102 38 370. Methanol is preferably removed by extraction from the top stream which comprises isoolefin or isobutene and is obtained in the first distillation step, and dimethyl ether and water are removed by distillation from the extracted isoolefin or isobutene.

When isobutene is prepared by the process according to the invention, it can be used, for example, to prepare methallyl chloride, methallyl sulphonates, methacrylic acid or methyl methacrylate. In particular, when both the methanol and the isobutene are removed from the bottom product, it may be advantageous to use both the methanol and the isobutene to prepare methyl methacrylate. Such a process for preparing methyl methacrylate is described, for example, in EP 1 254 887, to which reference is made explicitly.

The bottom product obtained in the first distillation step contains the compound of the formula I unconverted in the cleavage (for example MTBE) and the majority of the alcohol or water formed in the cleavage of the compound of the formula I. The bottom product can be recycled directly into the cleavage or else worked up in a second distillation step. The majority of the alcohol is preferably removed by distillation in a second distillation step from the bottom product of the first distillation step, and the remainder is recycled at least partly into the cleavage.

When columns are used in the process according to the invention, they may be provided with internals which are, for example, containing trays, rotating internals, random packings and/or structured packings.

In the case of the column trays, for example, the following types may be used:

Trays with bores or slots in the tray plate.

Trays with necks or chimneys which are covered by bubble-caps, caps or hoods.

Trays with bores in the tray plate, which are covered by movable valves.

Trays with special constructions.

In columns with rotating internals, the feed may, for example, be sprayed by rotating funnels or spread as a film on a heated tube wall with the aid of a rotor.

In the process according to the invention, as already stated, it is possible to use columns which have random packings of various packing materials. The packing materials may consist of almost all materials, especially of steel, stainless steel, copper, carbon, stoneware, porcelain, glass or plastics, and may have a wide variety of different shapes, especially the shape of spheres, rings with smooth or profiled surfaces, rings with inner elements or wall apertures, wire mesh rings, saddles and spirals.

Packings with regular/ordered geometry may consist, for example, of metal sheets or fabrics. Examples of such packings are Sulzer BX fabric packings made of metal or polymer, Sulzer Mellapak lamellae packings made of sheet metal, high-performance packings from Sulzer such as Mella-pakPlus, structured packings from Sulzer (Optiflow), Montz (BSH) and Kühni (Rombopak).

The isobutene obtained by the process according to the invention can be utilized for the purposes mentioned in the introduction.

Having generally described this invention, a further understanding can be obtained by reference to certain specific examples which are provided herein for purposes of illustration only, and are not intended to be limiting unless otherwise specified.

### EXAMPLES

### Example a

### Preparation of a Shaped Aluminosilicate Body

500 g of aluminosilicate powder (manufacturer: Grace Davison, Type: Davicat O 701, formal Al₂O₃ content: 13% by mass, formal SiO₂ content: 76% by mass, formal Na₂O content: 0.1% by mass, ignition loss at 850° C.: approx. 11%),

363 g of Disperal gel (formal Al₂O₃ content: 15.6%), (which was obtained by stirring 197 g of Disperal, a boehmite having a formal Al₂O₃ content of 77% by mass from Sasol Deutschland GmbH, into 803 g of 1.28% by mass aqueous nitric acid, subsequently stirring thoroughly, in the course of which the gel which forms was sheared constantly and thus kept in a free-flowing state, in a covered vessel at 60° C. for 3 h, cooling the gel to room temperature and replacement of any evaporated water), and

370 g of demineralized water (DM water) were initially mixed thoroughly with one another in an intensive mixer from Eirich. Subsequently, the mixture was pelletized in the intensive mixer from Eirich, to obtain uniformly round pellets with a diameter of approx. 1 to 3 mm within 30-40 minutes. The moist pellets were first dried in an air stream at 120° C. and then heated at 2 K/min to 550° C. and calcined in an air stream at this temperature for 10 h. The aluminosilicate pellets thus prepared contained, in a formal sense, 76% by mass of Al₂O₃ and 24% by mass of SiO₂. In addition, the catalyst prepared contained 0.12% by mass of sodium compounds (calculated as sodium oxide). The composition of the aluminosilicate pellets was calculated from the amount and the composition of the starting substances. The aluminosilicate pellets had a pore volume, determined by the above-described cyclohexane method, of 1.15 ml/g.

### Example b

### Preparation of a Shaped Catalyst (According to the Invention)

An impregnation solution having a magnesium content of 4.7% by mass was prepared from DM water and magnesium nitrate hexahydrate. The pH of this solution was 5.1. By means of vacuum impregnation, a screened-out fraction of the aluminosilicate support prepared in Example 1 (diameter: 1.0 mm-2.8 mm) was impregnated with the acidic magnesium nitrate solution. To this end, the pellets were introduced into a glass tube which was evacuated for about 30 min (water-jet pump vacuum of approx. 25 hPa). Subsequently, the impregnation solution was sucked in from the bottom up to above the upper edge of the solid-state bed. After an action time of about 15 minutes, the solution which had not been taken up by the support was discharged. The moist pellets were first dried to constant weight in an airstream at 140° C. and then heated to 450° C. at 3 K/min and calcined at this temperature for 12 h. The catalyst prepared consisted, in a formal sense, of 68% by mass of silicon dioxide, of 21% by mass of aluminium oxide and of 11% by mass of magnesium oxide. In addition, the catalyst prepared contained 0.11% by mass of sodium compounds (calculated as sodium oxide). The composition of the catalyst was calculated from the amount and the composition of the starting substances, and the impregnation solution which had run off. The amounts of sodium were part of the aluminosilicate used in Example 1. The pore volume, determined by the above-described cyclohexane method, was 1.1 ml/g.

### Example 1

### Comparative Example

In a stainless steel laboratory tubular reactor equipped with a jacket, experiments for the long-term testing of the catalyst were performed. The reactor was filled completely with a catalyst according to Example b having a particle size distribution from 1 to 2.8 mm. This corresponded to a catalyst mass of 283 g. For the heating of the reaction medium, Marlotherm SH from Sasol Olefins & Surfactants GmbH was used. The reactant was conducted through the tube and the Marlotherm SH through the jacket in cocurrent. The experiments were performed with establishment of the conditions listed in Table 1.

TABLE 1
Experimental conditions in Example 1

| | | |
|---|---|---|
| Marlotherm entrance temperature | [° C.] | 220 |
| Marlotherm exit temperature | [° C.] | 218-219 |
| Reactant entrance temperature | [° C.] | 220 |
| WHSV | [h⁻¹] | 9.6 |
| Pressure | [MPa] | 0.7 |

WHSV = weight hourly space velocity

Under these conditions, an initial value of 22% for the MTBE conversion and a minimum temperature in the tube interior of 164° C. were achieved. The selectivity for isobutene was 99.99%. Over 4000 hours, the conversion decreased to 2.5% with uniform selectivity.

### Example 2

### Inventive

In a stainless steel laboratory tubular reactor equipped with a jacket, experiments for the long-term testing of the catalyst were performed. The reactor was filled completely with a catalyst according to Example b having a particle size distribution from 1 to 2.8 mm. This corresponded to a catalyst mass of 283 g. For the heating of the reaction medium, Marlotherm SH from Sasol Olefins & Surfactants GmbH was used in co-current in the jacket. The experiments were performed with establishment of the conditions listed in Table 2.

TABLE 2
Experimental conditions in Example 2

| | | |
|---|---|---|
| Marlotherm entrance temperature | [° C.] | 250 |
| Marlotherm exit temperature | [° C.] | 250 |
| Reactant entrance temperature | [° C.] | 250 |
| WHSV | [h⁻¹] | 1.6 |
| Pressure | [MPa] | 0.7 |

Under these conditions, a starting value of 85% for the MTBE conversion and a minimum temperature in the tube interior of 218° C. were achieved. The selectivity for isobutene was 99.98%. Over 4000 hours, the conversion decreased to 70% with uniform selectivity.

The comparison of the results of Example 1 and 2 showed that the inventive procedure allowed the lifetime of the catalyst to be increased significantly.

German patent application DE 102006040433.5 filed Aug. 29, 2006, is incorporated herein by reference.

Numerous modifications and variations on the present invention are possible in light of the above teachings. It is therefore to be understood that within the scope of the appended claims, the invention may be practiced otherwise than as specifically described herein.

1. A continuous process for preparing an isoolefin having 4 to 6 carbon atoms, comprising:
cleaving a compound of the formula I
R₁—O—R₂ (I)
wherein R₁=a tertiary alkyl radical having 4 to 6 carbon atoms, and
R₂=H or an alkyl radical,
in a gas phase over a solid catalyst, in the temperature range of 200 to 400° C., at a pressure of 0.1 to 1.2 MPa, in a reactor which is equipped with a heating jacket and is heated with a liquid heat carrier,
wherein said cleavage is carried out in such a way that a temperature drop in the catalyst zone at any point in relation to the entrance temperature is less than 50° C.,
wherein (i) a reaction mixture in the reactor and (ii) the heat carrier in the jacket flow through the reactor in cocurrent, and
wherein a temperature difference of the heat carrier between a feed point to the reactor and an outlet from the reactor is adjusted to less than 40° C.

2. The process according to claim 1, wherein said compound of the formula I is selected from the group consisting of tert-butanol, methyl tert-butyl ether, ethyl tert-butyl ether, tert-amyl methyl ether and mixtures thereof.

3. The process according to claim 1, wherein a mixture of at least two compounds of the formula I is used.

4. The process according to claim 3, wherein the mixture of compounds of the formula I used is a mixture which comprises tert-butanol and methyl tert-butyl ether.

5. The process according to claim 1, wherein the temperature drop in the catalyst zone is less than 30° C.

6. The process according to claim 1, wherein the heat carrier is passed into the heating jacket of the reactor at a temperature which is 10 to 30° C. higher than the temperature of a reactant flowing into the reactor.

7. The process according to claim 1, wherein the solid catalyst is selected from the group consisting of metal oxides, mixed metal oxides, acids on metal oxide supports, metal salts and mixtures thereof.

8. The process according to claim 7, wherein solid catalyst has a mean particle size of 2 to 4 mm.

9. The process according to claim 1, wherein compound I is MTBE, and
wherein compound I is cleaved over a catalyst which comprises magnesium oxide, aluminium oxide and silicon oxide to give isobutene and methanol.

10. The process according to claim 1, which is performed in a tubular reactor, tube bundle reactor or plate reactor.

11. The process according to claim 1, wherein the process is performed in a tube bundle reactor.

12. The process according to claims 10 or 11, which is performed in a tube bundle reactor whose individual tubes have a length of 1 to 15 m.

13. The process according to claims 10 or 11, which is performed in a tube bundle reactor whose individual tubes have an internal diameter of 10 to 60 mm.

14. The process according to claims 10 or 11, which is performed in a tube bundle reactor whose individual tubes have a thickness of the tube wall of 1 to 4 mm.

15. The process according to claims 10 or 11, which is performed in a tube bundle reactor in which the tubes have a separation of 3 to 15 mm from one another.
